(19) United States
(12) Patent Application Publication
(10) Pub. No.: US 2026/0360476 A1
(43) Pub. Date: Nov. 12, 2026

(54) THREE-DIMENSIONAL MODELING OF MIXTURES FOR DETERMINING ACTIVATION LEVEL OF TASTE RECEPTORS

(71) Applicant: MARS, INCORPORATED, McLean, VA (US)

(72) Inventors: Scott Joseph MCGRANE, Melton Mowbray, Leicestershire (GB); Matthew Ronald GIBBS, Melton Mowbray, Leicestershire (GB); Boris KLEBANSKY, Melton Mowbray, Leicestershire (GB)

(73) Assignee: MARS, INCORPORATED, McLean, VA (US)

(21) Appl. No.: 19/130,832

(22) PCT Filed: Nov. 6, 2023
(86) PCT No.: PCT/US2023/036847

§ 371 Date: May 16, 2025

Related U.S. Application Data
(60) Provisional application No. 63/384,167, filed on Nov. 17, 2022.

(51) Int. Cl. G16C 20/30; A23K 50/40; G01N 33/50; G01N 33/566

(52) U.S. Cl. CPC G16C 20/30; A23K 50/40; G01N 33/5008; G01N 33/566

(57) ABSTRACT

Systems and methods for preparing a pet food by generating and using a model for predicting the response of a taste receptor are disclosed herein. In certain embodiments, the model is generated based on a function, where the function is approximated to a set of experimental data, and where the experimental data maps a combination of concentrations of taste modulators to a measured response of the taste receptor. Once generated, the model can be then used to predict expected receptor response for concentrations of taste modulators beyond those present in the experimental data.

### PRIORITY

This application claims the benefit, under 35 U.S.C. § 119(e), of U.S. Provisional Patent Application No. 63/384,167, filed 17 Nov. 2022, which is incorporated herein by reference.

### FIELD

The presently disclosed subject matter relates to systems and methods of generating models of compound mixtures that activate a taste receptor to a predetermined activity level. Computing systems can use such models for increasing the palatability of a food product by determining the concentrations of compounds for use in the manufacture of a food that activates certain taste receptors to a desired level.

### BACKGROUND

The activation of taste receptors, such as the umami T1R1/T1R3 taste receptor, to different degrees may be observed when applying varying concentrations of certain taste modulators to the receptor. Taste modulators that have been shown to have an effect on the umami receptor of cats include amino acids, nucleotides, and compounds that bind to the transmembrane domain of the receptor. Examples of such taste modulators include the amino acid alanine, the nucleotide guanosine monophosphate (or GMP), and the transmembrane binding compound N-(heptan-4-yl)benzo[d][1,3]dioxole-5-carboxamide. Simultaneously varying the concentration of pairs of these taste modulators (or taste modulator types) have been observed to produce a synergistic effect on the umami receptor of cats. For example, introducing a fixed amount of transmembrane compound at points on the half maximal effective concentration (EC50) curve of a nucleotide evinces an elevated response of the cat's umami receptor at certain combinations of the concentration of each taste modulator. Further, experimentation has shown that an even greater synergistic response of the umami receptor may be observed when concentrations of all three taste modulators (i.e., amino acid, nucleotide, and transmembrane compound) are varied simultaneously.

While experimental results have shown an elevated receptor response resulting from varying the concentrations of these three taste modulators, these experimental results have not previously given rise to a predictive model in which the response of the umami receptor may be determined, with a given level of confidence, based on the prior experimental response. That is, to date, no model has been developed in which an expected receptor response may be determined above and beyond the particular experimental outcomes in which a receptor response is physically measured based on specific amino acid, nucleotide, and transmembrane compound concentrations. Such a model would be a three-dimensional analog of the EC50 curve of a single taste modulator. That is, the model would provide for the interpolation and extrapolation of the expected response of the umami receptor beyond and between the specific data points that were physically measured. Among the uses of such a model would be as a tool for predicting receptor response of many different component mixtures, which would allow for the design of appropriate compound mixtures for taste testing, and for ultimate introduction of the mixtures as additives into food.

### SUMMARY OF THE INVENTION

The presently disclosed subject matter provides methods of generating a model for determining a response of a taste receptor to concentrations of at least a first taste modulator, a second taste modulator and a third taste modulator. In certain embodiments, the method comprises accessing a plurality of data points, each data point comprising at least one concentration level of the first taste modulator, one concentration level of the second taste modulator and one concentration level of the third taste modulator. The method further comprises, for each data point, determining a corresponding response of the taste receptor to the at least first, second and third taste modulators at the concentration levels according to each data point. The method further comprises selecting, for the model, a function of the concentration levels of the at least first, second and third taste modulators, the function comprising one or more unknown coefficients. The method further comprises determining the one or more unknown coefficients by fitting the function to the plurality of data points and the corresponding responses of the taste receptor. The method further comprises generating the model based on the function based on the determined one or more unknown coefficients.

The presently disclosed subject matter provides methods of preparing or enhancing palatability of a food product using a predictive model for determining a response of a taste receptor to concentrations of at least a first taste modulator, a second taste modulator and a third taste modulator. In certain embodiments, the method comprises selecting at least a first concentration of the first taste modulator, a second concentration of the second taste modulator and a third concentration of the third taste modulator. The method further comprises analyzing and transforming the at least first, second and third concentrations to derive a calculated response of a taste receptor using the predictive model, wherein the predictive model comprises a function of concentration levels of the at least first, second and third taste modulators, the function comprising one or more coefficients, and wherein the one or more coefficients are determined by fitting the function to a plurality of data points, each data point comprising predetermined concentration levels of the at least first, second and third taste modulators, and the corresponding responses of the taste receptor. The method further comprises preparing the food product comprising the first, the second and the third taste modulators at the first, the second and the third concentrations, respectively, when the calculated response of the taste receptor exceeds a threshold value.

In certain embodiments, the corresponding response of the taste receptor is determined by an in vitro assay. In certain embodiments, the in vitro assay comprises contacting the taste receptor with the at least first, second and third taste modulators at the concentration levels according to each data point and detecting a biological activity of the taste receptor.

In certain embodiments, the taste receptor is a human taste receptor, a feline taste receptor and/or a canine taste receptor. In certain embodiments, the taste receptor is an umami taste receptor, a kokumi taste receptor, and/or a sweet taste receptor.

In certain embodiments, the function is a sigmoid function. In certain embodiments, the function is a logistic function, a Gompetz sigmoid function, a trigonometric function, and/or a Hill equation. In certain embodiments, the one or more coefficients are determined by using nonlinear regression. In certain embodiments, the function has an approximation error of no more than 20%.

The presently disclosed subject matter provides food products prepared according to any method disclosed herein. In certain embodiments, the food product is a human food product or a pet food product. In certain embodiments, the pet food product is a feline pet food product or a canine pet food product. In certain embodiments, the pet food product is a wet pet food product. In certain embodiments, the pet food product is a dry pet food product.

The presently disclosed subject matter provides methods for determining concentrations of at least a first taste modulator, a second taste modulator and a third taste modulator in a food product. In certain embodiments, the method comprises determining at least a first concentration of the first taste modulator, a second concentration of the second taste modulator and a third concentration of the third taste modulator. The method further comprises analyzing and transforming the at least first, second and third concentrations to derive a calculated response of a taste receptor using a predictive model, wherein the predictive model comprises a function of concentration levels of the at least first, second and third taste modulators, the function comprising one or more coefficients, and wherein the one or more coefficients are determined by fitting the function to a plurality of data points, each data point comprising predetermined concentration levels of the at least first, second and third taste modulators, and the corresponding responses of the taste receptor. The method further comprises determining the first, the second and the third concentrations of the first, the second and the third taste modulators, respectively, for preparing the food product, when the calculated response of the taste receptor exceeds a threshold value.

The presently disclosed subject matter provides computer readable media, storing instructions that, when executed by a processor, cause a computer system to execute the steps of any method disclosed herein.

The presently disclosed subject matter provides systems for determining concentrations of at least a first taste modulator, a second taste modulator and a third taste modulator in a food product. In certain embodiments, the system comprises a processor and a memory that stores code. In certain embodiments, the stored code, when executed by the processor, causes the computer system to select at least a first concentration of the first taste modulator, a second concentration of the second taste modulator and a third concentration of the third taste modulator. The stored code, when executed by the processor, further causes the computer system to analyze and transform the at least first, second and third concentrations to derive a calculated response of a taste receptor using a predictive model, wherein the predictive model comprises a function of concentration levels of the at least first, second and third taste modulators, the function comprising one or more coefficients, and wherein the one or more coefficients are determined by fitting the function to a plurality of data points, each data point comprising predetermined concentration levels of the at least first, second and third taste modulators, and the corresponding responses of the taste receptor. The stored code, when executed by the processor, further causes the computer system to determine the first, the second and the third concentrations of the first, the second and the third taste modulators, respectively, for preparing the food product, when the calculated response of the taste receptor exceeds a threshold value.

The presently disclosed subject matter provides systems for determining concentrations of at least a first taste modulator, a second taste modulator and a third taste modulator in a food product. In certain embodiments, the system comprises a processor, a user interface, and a memory that stores code. In certain embodiments, the stored code, when executed by the processor, causes the computer system to select at least a first concentration of the first taste modulator, a second concentration of the second taste modulator and a third concentration of the third taste modulator. The stored code, when executed by the processor, further causes the computer system to analyze and transform the at least first, second and third concentrations to derive a calculated response of a taste receptor using a predictive model, wherein the predictive model comprises a function of concentration levels of the at least first, second and third taste modulators, the function comprising one or more coefficients, and wherein the one or more coefficients are determined by fitting the function to a plurality of data points, each data point comprising predetermined concentration levels of the at least first, second and third taste modulators, and the corresponding responses of the taste receptor. The stored code, when executed by the processor, further causes the computer system to determine the first, the second and the third concentrations of the first, the second and the third taste modulators, respectively, for preparing the food product, when the calculated response of the taste receptor exceeds a threshold value. The stored code, when executed by the processor, further causes the computer system to display the determination of the first, the second, and the third concentrations on the user interface.

### BRIEF DESCRIPTION OF THE DRAWINGS

FIG. 1 depicts synergy plots in which different concentrations of alanine and GMP are applied to the umami receptor of a cat.

FIG. 2 depicts synergy plots in which different concentrations of alanine and GMP and a constant concentration of N-(heptan-4-yl)benzo[d][1,3]dioxole-5-carboxamide are applied to the umami receptor of a cat.

FIG. 3 is a flow diagram of a method that determines a model that is adapted to predict the level of response of a receptor based on concentrations of a plurality of taste modulators, according to one or more embodiments.

FIG. 4 depicts luminosity plots that show receptor response to combinations of GMP, alanine, and N-(heptan-4-yl)benzo[d][1,3]dioxole-5-carboxamide.

FIG. 5 depicts comparative luminosity plots that show the actual receptor response to combinations of GMP, alanine, and Benzyl-L-phenylalanine methyl ester HCl and receptor responses predicted by a model to combinations of these taste modulators, according to embodiments.

FIG. 6 is a flow diagram that depicts a method for formulating a pet food based on an optimal cost combination of taste modulators that give rise to a desired receptor response, according to one or more embodiments.

FIG. 7 is a flow diagram that depicts a method for generating a model for determining a response of a taste receptor to concentrations of at least a first taste modulator, a second taste modulator and a third taste modulator.

FIG. 8 is a flow diagram that depicts a method for preparing or enhancing palatability of a food product using a predictive model for determining a response of a taste receptor to concentrations of at least a first taste modulator, a second taste modulator and a third taste modulator.

FIG. 9 is a flow diagram that depicts a method for determining concentrations of at least a first taste modulator, a second taste modulator and a third taste modulator in a food product.

FIG. 10 illustrates an example computer system.

FIG. 11 is a graph that compares experimental results of applying varying concentrations of three taste modulators to the umami receptor of a cat with results of generated by three sigmoid functions modeled according to one or more embodiments.

FIGS. 12a-12c depict graphs that compare experimental results of applying varying concentrations of three taste modulators to the umami receptor of a cat with results generated by a predictive model, according to one or more embodiments.

### DETAILED DESCRIPTION

### 1. Definitions

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “food product” refers to an ingestible product, such as, but not limited to, human food, animal (pet) foods, and pharmaceutical compositions.

The term “pet food” or “pet food composition” or “pet food product” or “final pet food product” means a product or composition that is intended for consumption by, and provides certain nutritional benefit to a companion animal, such as a cat, a dog, a guinea pig, a rabbit, a bird or a horse. For example, but not by way of limitation, the companion animal can be a “domestic” dog, e.g., Canis lupus familiaris. In certain embodiments, the companion animal can be a “domestic” cat such as Felis domesticus. A “pet food” or “pet food composition” or “pet food product” or “final pet food product” includes any food, feed, snack, food supplement, liquid, beverage, treat, toy (chewable and/or consumable toys), meal substitute or meal replacement.

As used herein, “taste” refers to a sensation caused by activation or inhibition of receptor cells in a subject's taste buds. In certain embodiments, taste can be selected from the group consisting of sweet, sour, salty, bitter, kokumi, free fatty acid and umami. In certain embodiments, a taste is elicited in a subject by a “tastant” or “taste modulator.” In certain embodiments, a tastant is a synthetic tastant. In certain embodiments, the tastant is prepared from a natural source.

In certain embodiments, “taste” can include kokumi taste. See, e.g., Ohsu et al., J. Biol. Chem., 285(2): 1016-1022 (2010), the contents of which are incorporated herein by reference. In certain embodiments, kokumi taste is a sensation caused by activation or inhibition of receptor cells in a subject's taste buds, for example the receptor CaSR, and is separate than other tastes, for example, sweet, salty, and umami tastes, although it can act as a taste enhancer for these tastes.

As used herein, “taste profile” refers to a combination of tastes, such as, for example, one or more of a sweet, sour, salt, bitter, umami, kokumi and free fatty acid taste. In certain embodiments, a taste profile is produced by one or more tastant that is present in a composition at the same or different concentrations. In certain embodiments, a taste profile refers to the intensity of a taste or combination of tastes, for example, a sweet, sour, salt, bitter, umami, kokumi and free fatty acid taste, as detected by a subject or any assay known in the art. In certain embodiments, modifying, changing or varying the combination of tastants in a taste profile can change the sensory experience of a subject.

As used herein, the terms “modulates” or “modifies” refers an increase or decrease in the amount, quality or effect of a particular activity of a receptor and/or an increase or decrease in the expression, activity or function of a receptor. “Modulators,” as used herein, refer to any inhibitory or activating compounds identified using in silico, in vitro and/or in vivo assays for, e.g., agonists, antagonists and their homologs, including fragments, variants and mimetics.

As used herein “admixing,” for example, “admixing the flavor composition or combinations thereof of the present application with a food product,” refers to the process where the flavor composition, or individual components of the flavor composition, is mixed with or added to the completed product or mixed with some or all of the components of the product during product formation or some combination of these steps. When used in the context of admixing, the term “product” refers to the product or any of its components. This admixing step can include a process selected from the step of adding the flavor composition to the product, spraying the flavor composition on the product, coating the flavor composition on the product, suspending the product in the flavor composition, painting the flavor composition on the product, pasting the flavor composition on the product, encapsulating the product with the flavor composition, mixing the flavor composition with the product and any combination thereof. The flavor composition can be a liquid, emulsion, dry powder, spray, paste, suspension and any combination thereof.

As used herein, “synergy,” or “synergistic response” may refer to an effect produced by two or more individual components in which the total effect produced by these components, when utilized in combination, is greater than the sum of the individual effects of each component acting alone.

As used herein, “palatability” can refer to the overall willingness of a human and/or an animal to eat a certain food product. Increasing the “palatability” of a pet food product can lead to an increase in the enjoyment and acceptance of the pet food by the companion animal to ensure the animal eats a “healthy amount” of the pet food. The term “healthy amount” of a pet food as used herein refers to an amount that enables the companion animal to maintain or achieve an intake contributing to its overall general health in terms of micronutrients, macronutrients and calories, such as set out in the “Mars Petcare Essential Nutrient Standards.” In certain embodiments, “palatability” can mean a relative preference of an animal for one food product over another. For example, when an animal shows a preference for one of two or more food products, the preferred food product is more “palatable,” and has “enhanced palatability.” In certain embodiments, the relative palatability of one food product compared to one or more other food products can be determined, for example, in side-by-side, free-choice comparisons, e.g., by relative consumption of the food products, or other appropriate measures of preference indicative of palatability. Palatability can be determined by a standard testing protocol in which the animal has equal access to both food products such as a test called “two-bowl test” or “versus test.” Such preference can arise from any of the animal's senses, but can be related to, inter alia, taste, aftertaste, smell, mouth feel and/or texture.

### 2. Methods and Systems

Methods and systems for generating and using a computer-based model for determining receptor response (such as the response of the umami receptor) based on varying concentrations of three or more taste modulators are disclosed herein. In particular embodiments, a computing system can derive the model from a functional assay of the umami receptor, where the response of the receptor is measured in response to varying concentrations of at least an amino acid, a nucleotide, and a compound that binds to the transmembane domain of the umami receptor (referred to herein as a transmembrane compound). Although the models described herein were developed through activation of the cat umami receptor with amino acids, nucleotides and transmembrane compounds, the models described herein are applicable to other taste receptors, for example, human taste receptors, that can be activated by one, two, three or more receptor activating compounds.

The functional assay gives rise to a plurality of data points, which may be represented as a data structure, such as an array, matrix, vector, lookup table or combinations thereof, that can be stored in the memory of a computer, or on a peripheral or network-attached storage device. In particular embodiments, the data points correspond to a concentration of an amino acid, a concentration of a nucleotide, and a concentration of a transmembrane compound. As previously mentioned, the amino acid, nucleotide, and transmembrane compound may be alanine, GMP and N-(heptan-4-yl)benzo[d][1,3]dioxole-5-carboxamide, respectively. Further, the data used to derive the model includes an indication of a level of response of a receptor when these concentrations are applied thereto.

The experimental cat umami receptor response to mixtures of alanine, GMP, and N-(heptan-4-yl)benzo[d][1,3]dioxole-5-carboxamide demonstrates synergistic positive allosteric modulator action between these taste modulators. Structurally, these taste modulators interact with three different positions of the umami receptor. Alanine binds to an amino-acid binding site in the venus flytrap domain of the T1R1 receptor subunit near the hinge of the external flytrap domain. GMP binds to a nucleotide binding site in the venus flytrap domain of T1R1. Finally, N-(heptan-4-yl)benzo[d][1,3]dioxole-5-carboxamide binds to the seven-transmembrane domain of T1R1. Examination of experimental data reveals that any pairwise combination, as well as the combination of all three taste modulators, demonstrates a synergistic effect with respect to activation of the receptor. That is, according to the experimental data, the level of response of the receptor from varying the combination of the taste modulators is greater than the linear sum of the response of the receptor if each taste modulator is varied individually. It is noted here that the response of the umami receptor to changes in concentrations of one or more taste modulators may be measured as a certain degree of luminescence.

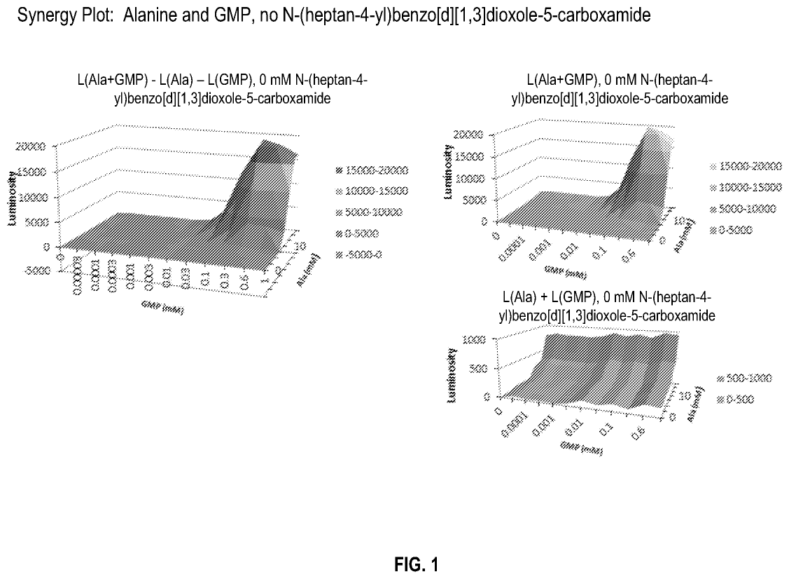

For example, FIG. 1 depicts synergy plots in which different concentrations of alanine and GMP are applied to the umami receptor of a cat, while N-(heptan-4-yl)benzo[d][1,3]dioxole-5-carboxamide is not applied to the receptor. The plot in the left panel depicts the difference in the response of the receptor when both alanine and GMP are varied and the sum of the responses of the receptor when each of alanine and GMP are individually varied. Concentration of GMP (in millimolar, or mM) varies along the x-axis, while concentration of alanine (also in mM) varies along the y-axis. Receptor response (depicted as luminosity) is measured on the z-axis. As shown, the difference between the “combined” response and the sum of the individual responses is most notable when the concentrations of GMP and alanine are increased to 06, and 10 mM, respectively. This difference represents the synergy of the combination.

This synergistic response is also evident when viewing separately the receptor response of the combination of GMP and alanine (as depicted by the top right-hand plot in FIG. 1) and the sum of the individual receptor responses to GMP and alanine (which is depicted in the bottom right-hand plot in FIG. 1).

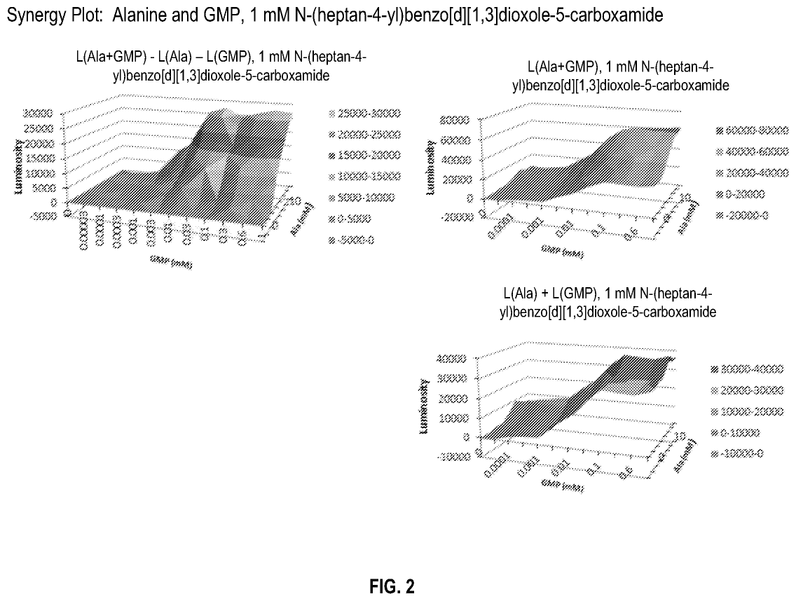

Experimental data has shown that even greater synergies in terms of receptor response can be realized by introducing concentrations of other taste modulators. For example, FIG. 2 depicts synergy plots in which, in contrast to the case depicted in FIG. 1, the N-(heptan-4-yl)benzo[d][1,3]dioxole-5-carboxamide taste modulator is also applied to the receptor. Thus, in the left-hand plot, concentrations of GMP and alanine are again varied along the x- and y-axes, respectively. In this case, the concentration of N-(heptan-4-yl)benzo[d][1,3]dioxole-5-carboxamide is held constant at 1 mM. As shown in the figure, the difference between the “synergistic” response of the receptor (depicted again as luminosity level) and the sum of the individual responses of the receptor to variations in each of the alanine and GMP taste modulators, is even greater. In the case of FIG. 2, a higher synergistic receptor response is displayed at an approximate concentration of 0.03 mM of GMP and less than 10 mM of alanine.

Further, still referring to FIG. 2, the plots in the top-right and bottom-right panels depict, respectively, the receptor response to varying the concentrations of GMP and alanine (while holding constant the concentration of N-(heptan-4-yl)benzo[d][1,3]dioxole-5-carboxamide at 1 mM), and the sum of the receptor responses to individually applying and varying the concentration GMP and alanine (again, while applying a constant concentration of 1 mM of N-(heptan-4-yl)benzo[d][1,3]dioxole-5-carboxamide). A comparison of the top-right and bottom-right plots shows at that the synergistic response of the combination has approximately double the luminescence of that of the sum of the individual responses. Furthermore, it should be noted that still greater synergistic responses can be realized by varying the concentration of N-(heptan-4-yl)benzo[d][1,3]dioxole-5-carboxamide.

Based on the empirical results depicted in FIGS. 1 and 2, it is advantageous to formulate a predictive model that can be used to forecast the expected response of a receptor to varying concentrations of three taste modulators. It should be noted that the methodology disclosed herein may be applied to variations of more than three taste modulators and is designed to capture synergies among any type of agonist and any number of allosteric modulators. In one or more embodiments, the computing system can develop such a model by selecting a functional type for the model and generating a closed-form equation that has three variables, where each variable corresponds to a concentration of a component of a mixture that is applied to a taste receptor. Upon substituting values for each of the component concentrations, the model generates an expected physical response of the receptor for the concentration levels. Alternatively, the model can also accept a desired receptor response and may be used to generate combinations of concentrations of components that give rise to the desired receptor response.

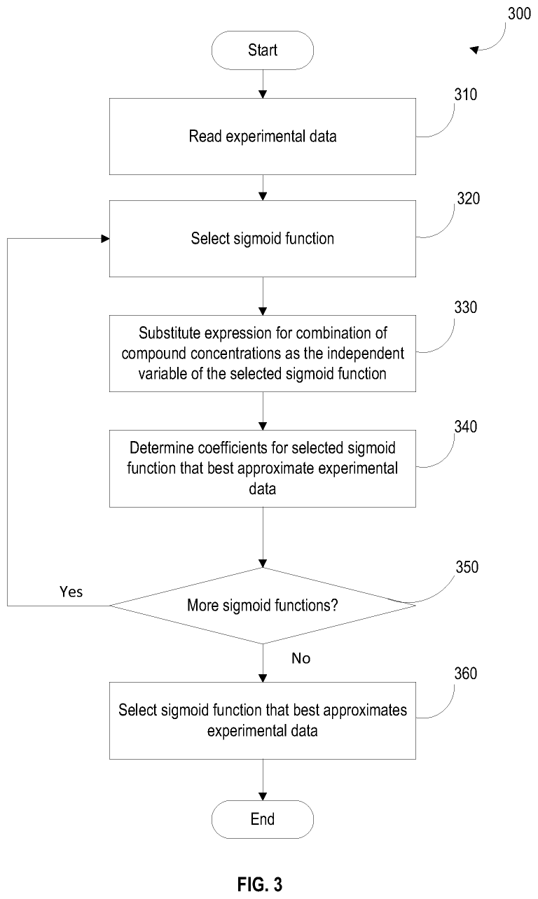

FIG. 3 is a flow diagram for a method 300 that determines a computer-based model that is adapted to predict the level of response of a receptor, such as the umami receptor, based on concentrations of a plurality of taste modulators, according to one or more embodiments. According to embodiments, the model is capable of being implemented in computer software on a desktop, laptop, notebook, handheld, or server-class computing device. Further, method 300 is, in embodiments, implemented in computer software and is capable of being executed on similar computing devices.

Method 300 begins at step 310, at which the computing system reads experimental data from a database or file. As noted earlier, experimental data comprises data points that define a particular response of a receptor when certain concentrations of different compounds are applied thereto. For example, each data point may be represented as an n-tuple, in which the first n−1 elements represent concentrations of different compounds that have been applied to the umami receptor of a cat. The n-th element then represents the response level of the receptor to the given concentrations. This response level can be represented visually as a level of luminosity.

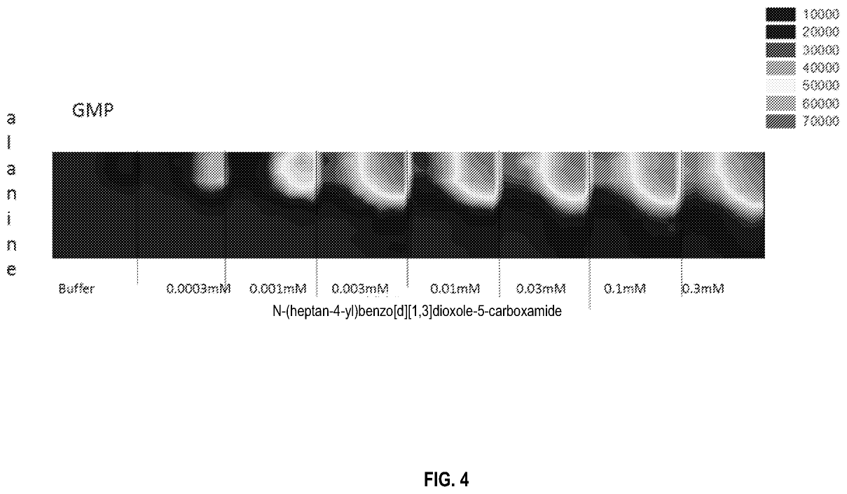

FIG. 4 depicts an example of experimental results for combinations of GMP, alanine, and N-(heptan-4-yl)benzo[d][1,3]dioxole-5-carboxamide. The figure depicts eight panels, each of which corresponds to a concentration of N-(heptan-4-yl)benzo[d][1,3]dioxole-5-carboxamide. Thus, the first panel shows a zero concentration of N-(heptan-4-yl)benzo[d][1,3]dioxole-5-carboxamide, the second shows a concentration of 0.0003 mM, the third panel shows a concentration of 0.001 mM, and so on, up to the eighth panel, which shows a concentration of 0.3 mM. Within each panel, the concentrations of GMP and alanine are varied. GMP concentration is varied along the horizontal axis, while alanine is varied along the vertical. In this experiment, GMP concentration is varied uniformly from 0 to 1 mM, while alanine concentration is varied from 0 to 100 mM. The response of the receptor is depicted as the luminosity (represented by the shading of each panel at the several interior points). Thus, as shown in the figure, the first panel (at which concentration of N-(heptan-4-yl)benzo[d][1,3]dioxole-5-carboxamide is zero, variations of the concentrations of GMP and alanine do not result in any receptor response. However, when the concentration of N-(heptan-4-yl)benzo[d][1,3]dioxole-5-carboxamide is increased (e.g., to 0.0003 mM, 0.001 mM, and so on), the same variations in the concentrations of GMP and alanine result in higher response levels of the receptor. Thus, in the third panel (where the concentration of N-(heptan-4-yl)benzo[d][1,3]dioxole-5-carboxamide is 0.001 mM, a receptor response having a luminosity of 60,000 is observed as the concentrations of GMP and alanine are increased. However, in the eighth panel (where the concentration of N-(heptan-4-yl)benzo[d][1,3]dioxole-5-carboxamide is 0.3 mM), the receptor response is greater as the concentrations of GMP and alanine are increased (i.e., the response is represented by a luminosity of roughly 70,000).

As shown in FIG. 4, data points between the measured concentrations of the three taste modulators are linearly interpolated to determine a level of luminescence for concentrations for which no measurement was obtained. However, linear interpolation is sub-optimal in predicting the luminescent response of an umami receptor to an arbitrary combination of concentrations of the three taste modulators (i.e., alanine, GMP, and N-(heptan-4-yl)benzo[d][1,3]dioxole-5-carboxamide). This can be inferred from the non-linearity of the luminescent response that is shown in the data plotted in FIG. 4. This evinces a need for a more reliable and predictive model that replicates experimental results. Such a predictive model also provides a basis for describing synergistic effects, not only for the particular mixture of compounds depicted (i.e., alanine, GMP, and N-(heptan-4-yl)benzo[d][1,3]dioxole-5-carboxamide), but also for other synergistic combinations of tastants.

The experimental results shown in FIG. 4 can be stored as a plurality of 4-tuples in a database or file stored on a data storage device. For example, a sample data “point” can be represented as [0.3 mM, 1 mM, 100 mM, and 70000], where 0.3 mM is the concentration of N-(heptan-4-yl)benzo[d][1,3]dioxole-5-carboxamide, 1 mM is the concentration of GMP, 100 mM is the concentration of alanine, and 70,000 is the corresponding luminosity (or receptor response level). A plurality of data points can be represented in this way and stored in a database. Typically, experimental data consists of 1,000 data points, which represents combinations of 10 concentrations of each compound (i.e., 10×10×10 probes are required, resulting in 1,000 measured receptor responses).

It should be noted that the database or file storing the experimental data may be populated using various data population methods. For example, an end user (such as an experimenter) may access a computer interface (such as a graphical user interface (GUI) or command-line interface (CLI)) and manually enter the data points (corresponding to the concentrations of compounds) and the corresponding measured luminescence of the receptors. Further, in other embodiments, the data points and luminescence may be transmitted as a file or data stream to a receiving process on a computer, where the receiving process automatically populates the database. In still other embodiments, an apparatus configured to conduct the measurement of luminescence of the umami receptor based on detected concentrations of taste modulators is further configured to automatically transmit the concentrations and luminescence data to a receiving process for subsequent entry into the database or file. Moreover, a process that measures the luminescence of the umami receptor may itself be configured to directly access and populate the database or file.

It should also be noted that the database may be implemented as a single table, multiple relational database tables, or one or more arrays, hash maps, or linked lists. Other data structures capable of storing three-dimensional data are also within the scope of this disclosure.

Referring back to FIG. 3, after reading the experimental results at step 310, method 300 proceeds to step 320. At step 320, the computing system selects one sigmoid function from among a plurality of sigmoid functions to be evaluated to approximate the experimental data.

A functional type for modeling receptor response is one with a sigmoidal (or “S”) shape, and many biological processes can be modeled using this shape. Several sigmoid functions are used in biological modeling. Among the functions used are the logistic function, the Gompetz sigmoid function, trigonometric functions, and the Hill equation.

The logistic function can be represented as:

$$f ( x ) = \frac{1}{1 + {e}^{- x}} .$$

The Gompetz sigmoid function can be represented as f(x)=ae^{be}^{^{−ex}}, where a, b, and c may be varied to change, respectively, asymptote, displacement, and growth rate. A trigonometric function that evinces a sigmoid shape is the hyperbolic tangent function, or tan h (x). Finally, the Hill equation can be represented as

$$f ( x ) = \frac{{x}^{n}}{1 + {x}^{n}} .$$

Further, other sigmoid functions can be equation can be represented as obtained using linear combinations of other known sigmoid functions, as well as by multiplication and/or superposition of known sigmoid functions.

Thus, according to embodiments, any one of the aforementioned functions (or, indeed, another sigmoid function) may be selected. Such functions may be stored, for example, as equations in a database, where parameter values (e.g., the value of “n” in the Hill equation) are able to be varied.

Having selected a sigmoid function, method 300 proceeds to step 330. At step 330, the computing system substitutes an expression representing a combination of compound concentrations as the independent variable of the selected sigmoid function. In one or more embodiments, an expression for the concentrations of the compounds is defined as the sum of the products of the compound concentrations and the binding constants, as shown by the following expression:

aₓx+a_{y}y+a_{z}z+a_{xy}xy+a_{xz}xz+a_{yz}yz+a_{xyz}xyz

In this expression, x, y, and z are the concentrations of each compound (e.g., alanine, GMP and N-(heptan-4-yl)benzo[d][1,3]dioxole-5-carboxamide, respectively), and the an coefficients are unknown binding constants. Thus, assuming that the selected sigmoid function is the Hill equation, after substitution of the above expression, the sigmoid function can be represented as:

$$f ( x , y , z ) = \frac{{( {a}_{x} x + {a}_{y} y + {a}_{z} z + {a}_{xy} xy + {a}_{xz} xz + {a}_{yz} yz + {a}_{xyz} xyz )}^{n}}{1 + {( {a}_{x} x + {a}_{y} y + {a}_{z} z + {a}_{xy} xy + {a}_{xz} xz + {a}_{yz} yz + {a}_{xyz} xyz )}^{n}}$$

Similarly, if the selected sigmoid function is the logistic function, then the sigmoid function can be represented as:

$$f ( x , y , z ) = \frac{1}{1 + {e}^{- ( {a}_{x} x + {a}_{y} y + {a}_{z} z + {a}_{xy} xy + {a}_{xz} xz + {a}_{yz} yz + {a}_{xyz} xyz )}}$$

Once the expression for the compound concentrations is substituted into the selected sigmoid function, method 300 proceeds to step 340. At step 340, the computing system determines values for the unknown coefficients in the sigmoid function. For example, in the case of the Hill equation, the unknown coefficients are the binding constants aₓ, a_{y}, a_{z}, a_{xz}, a_{yz}, and a_{xyz}, as well as the exponent n. Similarly, for the logistic function, the unknown coefficients are the aforementioned binding constants. For the Gompetz sigmoid function, the unknown coefficients are the binding constants, as well as asymptote, displacement, and growth rate.

In particular embodiments, the computing system can determine the unknown coefficients of the updated sigmoid function generated at step 330 using nonlinear regression. That is, the coefficients of the function f(x, y, z) are selected to better approximate (or “fit”) the experimental data that was read in at step 310. In this manner, the difference between the simulated luminosity values generated by the sigmoid function and the actual luminosity levels specified in the experimental data is reduced.

At step 340, the computing system makes successive approximations to reduce or minimize the error between the sigmoid function and the experimental data. For example, assuming that the selected sigmoid function is the Hill equation, the value of the exponent (i.e., “n”) is varied in increments of 0.1 from 0 to 2. The values of the binding constants (i.e., aₓ, a_{y}, a_{z}, a_{xz}, a_{yz}, and a_{xyz}) are initially set to zero and are similarly varied according to predetermined increments. As the coefficients are varied, the computing system computes the approximation error for each set of coefficients. The set of coefficients that results in the lowest error is then retained. As an example, when the selected sigmoid function is the Hill equation, the lowest approximation error for an exemplary set of data has been shown to be approximately 10 percent. In certain embodiments, a threshold of approximation error must be met for the function to be considered, or a different function will be used. In certain embodiments, the threshold of approximation error is no more than about 30%, about 25%, about 20%, about 15%, about 10%, about 5% or less. In certain embodiments, the threshold of approximation error is between about 5% and about 30%, between about 5% and about 25%, between about 5% and about 20%, between about 10% and about 30%, or between about 10% and about 20%.

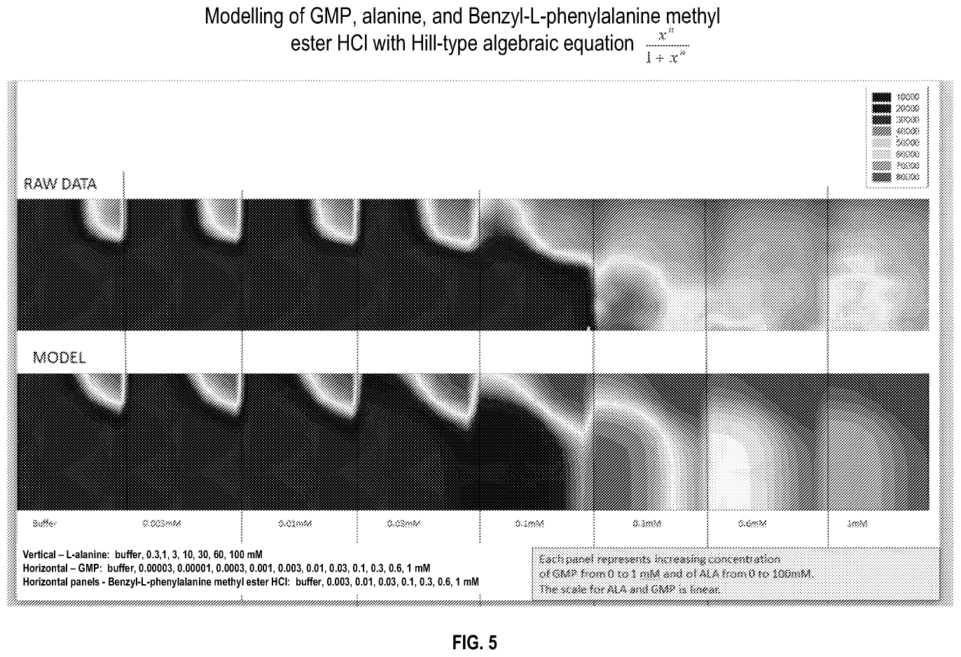

This approximation error is shown visually in FIG. 5. As shown in the figure, the “raw” experimental data comprises luminosity levels (i.e., umami receptor response) for varying concentrations of GMP, alanine and Benzyl-L-phenylalanine methyl ester HCl, where the latter is a synthetic taste modulator capable of binding to the umami receptor. The top row of panels depicts the receptor response as the concentration of Benzyl-L-phenylalanine methyl ester HCl is increased in discrete increments from 0 to 1 mM. Furthermore, similar to the case shown in FIG. 4, the concentration of GMP is varied from 0 to 1 mM and the concentration of alanine is varied from 0 to 100 mM.

The bottom row of panels in FIG. 5 depicts the predicted receptor response generated by the Hill equation model, where the concentrations of Benzyl-L-phenylalanine methyl ester HCl, GMP, and alanine are substituted for the variables x, y, and z, respectively. The luminosity plots depicted in FIG. 5 represent only one set of coefficients for the Hill model (i.e., for one combination of binding constants a and exponent n). That is, for different combinations of binding constants and/or exponent values, the plots depicted in the bottom panel of FIG. 5 would change, evincing a better or worse approximation than the one shown in the figure. With reference to method 300, step 340, the computing system selects and stores the sigmoid function having the best approximation (i.e., the lowest error). As an example, in the case of the Hill equation, the best approximation error for an exemplary set of experimental data combining concentrations of Benzyl-L-phenylalanine methyl ester HCl, GMP, and alanine has been determined to be 9.5 percent. In this case, the determined coefficients are as follows: aₓ=0; a_{y}=0; a_{z}=2.68; a_{xy}=0.09, a_{yz}=0.07; a_{xz}=12; a_{xyz}=5; and n=1.41. Thus, the resulting modified Hill equation for this case would be represented as:

$$f ( x , y , z ) = \frac{{( 2.68 z + 0.09 xy + 12 xz + 0.07 yz + 5 xyz )}^{1.41}}{1 + {( 2.68 z + 0.09 xy + 12 xz + 0.07 yz + 5 xyz )}^{1.41}}$$

where x, y, and z represent the concentrations of GMP, alanine, and Benzyl-L-phenylalanine methyl ester HCl, respectively.

Referring back to FIG. 3, method 300 then proceeds to step 350. At step 350, the computing system makes a determination whether any sigmoid functions remain to be evaluated to approximate the experimental data. For example, assuming that the first selected sigmoid function was the Hill equation, and that the method is adapted to further evaluate sigmoid functions having the hyperbolic tangent, logistic, or Gompetz forms, then the computing system would determine, at step 350, that these additional sigmoid functions remain to be evaluated. If method 300 is adapted to only evaluate a single sigmoid function, or if all sigmoid functions have been evaluated, then the computing system would determine that there are no more sigmoid functions to be evaluated.

If there are more sigmoid functions to be evaluated, then method 300 proceeds back to step 320, where the computing system selects a next sigmoid function. Method 300 then proceeds through steps 330 and 340 for the next selected sigmoid function.

However, if there are no more sigmoid functions to be selected, then method 300 proceeds to step 360. At step 360, the computing system selects the sigmoid function having the lowest approximation error among all the evaluated sigmoid functions as the model. For example, as previously mentioned, the Hill equation evinces the lowest approximation error of around 10 percent for an exemplary set of experimental data. By contrast, the logistic function and hyperbolic tangent functions have lowest approximation errors of about 25 percent for the same exemplary set of experimental data. Thus, in the case, the Hill equation (using the coefficients determined at step 340) would be selected as the model.

After the sigmoid function for the model is selected at step 360, method 300 terminates.

Once a model has been computed based on experimental data, the model may then be used to determine a predicted receptor response for a given concentration of taste modulators. Alternatively, the model may be used to determine an unknown concentration of one taste modulator, given specified concentrations for the other taste modulators, as well as a specific desired receptor response. Further, the model may be used to determine a set of combinations of compounds that give rise to at least a threshold receptor response. This use is advantageous because it allows for a determination of the most cost-effective concentrations of compounds that result in an acceptable activation level of the receptor. In other words, if one of the compounds is particularly expensive, it would be advantageous to decrease that compound's concentration, while appropriately altering the concentrations of the other compounds to maintain the desired receptor response. As noted earlier, the model may be implemented on a general-purpose computer. Inputs to the model (i.e., concentrations of compounds or a desired response level for the receptor) may be provided to the model using any suitable computer interface, for example, a graphical user interface, a command line interface, pen-based input, voice recognition, or any other means by which data may be provided to a computer program.

The execution of the model may include deployment of one or more machine-learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification, or the like. Any suitable type of training may be used, e.g., stochastic, gradient boosted, random seeded, recursive, epoch or batch-based, etc.

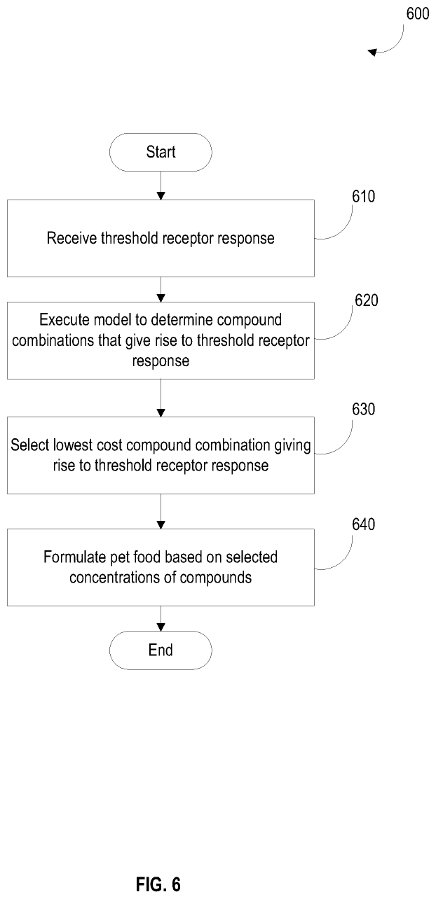

FIG. 6 is a flow diagram that depicts a method 600 for formulating a pet food based on an optimal cost combination of taste modulators that give rise to a desired receptor response, according to one or more embodiments. Method 600 begins at step 610, where the computing system receives a desired level of receptor response. The desired receptor response is represented, in particular embodiments, as a particular threshold luminosity level, as shown in FIGS. 1 and 2. Further, the desired receptor response can be entered into the model by an end user using a computer interface.

Next, at step 620, the computing system executes the model to determine all combinations of taste modulators (e.g., GMP, alanine, and Benzyl-L-phenylalanine methyl ester HCl) that result in at least the received desired receptor response. To achieve this, the computing system executes the model numerous times, where various concentrations of each taste modulator are chosen. In some embodiments, each taste modulator concentration is varied in increments within a range of values up to a maximum value (such as, for example, 1 mM for Benzyl-L-phenylalanine methyl ester HCl), while each other taste modulator concentration is held constant. This process is repeated for each taste modulator, until a predicted receptor response has been computed for all possible combinations of taste modulator concentrations. If a receptor response meets the response threshold entered by the user, then the corresponding combination of taste modulator concentrations is saved and/or output to the user. Otherwise, if the receptor response does not meet the threshold response level, then the corresponding combination of taste modulator concentrations is dismissed.

Next, at step 630, the computing system determines an optimal cost combination of taste modulator concentrations. To achieve this, method 600 can be adapted to access cost data, which specifies the unit cost of each taste modulator. The computing system would then examine each of the combinations of taste modulator concentrations saved and/or output at step 620 and calculate the cost of each concentration based on the cost data. The method is adapted to then select the lowest cost combination of taste modulator concentrations that meets or exceeds the threshold desired receptor response.

Next at, at step 640, a pet food is formulated including the selected concentrations of taste modulators at step 630.

After step 640, method 600 terminates.

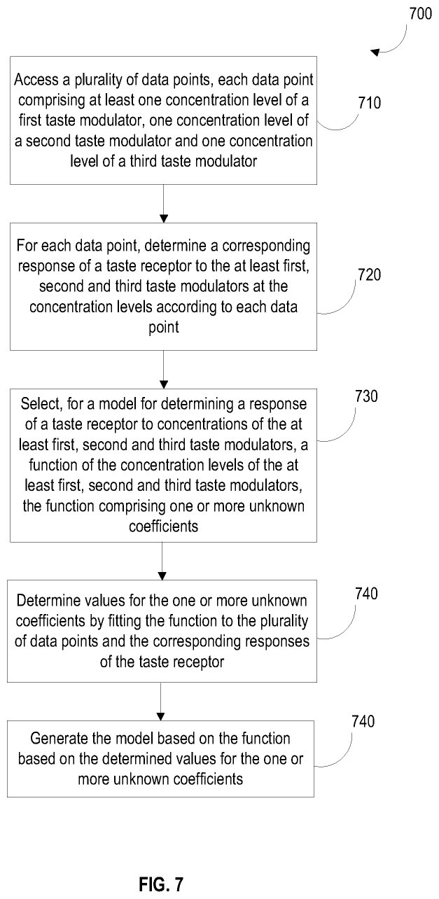

FIG. 7 is a flow diagram that depicts a method 700 for generating a model for determining a response of a taste receptor to concentrations of at least a first taste modulator, a second taste modulator and a third taste modulator. The method may begin at step 710, where the computing system can access a plurality of data points, each data point comprising at least one concentration level of the first taste modulator, one concentration level of the second taste modulator and one concentration level of the third taste modulator. At step 720, the computing system can, for each data point, determine a corresponding response of the taste receptor to the at least first, second and third taste modulators at the concentration levels according to each data point. At step 730, the computing system can select, for the model, a function of the concentration levels of the at least first, second and third taste modulators, the function comprising one or more unknown coefficients. At step 740, the computing system can determine values for the one or more unknown coefficients by fitting the function to the plurality of data points and the corresponding responses of the taste receptor. At step 750, the computing system can generate the model based on the function based on the determined values for the one or more unknown coefficients. Particular embodiments may repeat one or more steps of the method of FIG. 7, where appropriate. Although this disclosure describes and illustrates particular steps of the method of FIG. 7 as occurring in a particular order, this disclosure contemplates any suitable steps of the method of FIG. 7 occurring in any suitable order. Moreover, although this disclosure describes and illustrates an example method for generating a model for determining a response of a taste receptor to concentrations of at least a first taste modulator, a second taste modulator and a third taste modulator including the particular steps of the method of FIG. 7, this disclosure contemplates any suitable method for generating a model for determining a response of a taste receptor to concentrations of at least a first taste modulator, a second taste modulator and a third taste modulator including any suitable steps, which may include all, some, or none of the steps of the method of FIG. 7, where appropriate. Furthermore, although this disclosure describes and illustrates particular components, devices, or systems carrying out particular steps of the method of FIG. 7, this disclosure contemplates any suitable combination of any suitable components, devices, or systems carrying out any suitable steps of the method of FIG. 7.

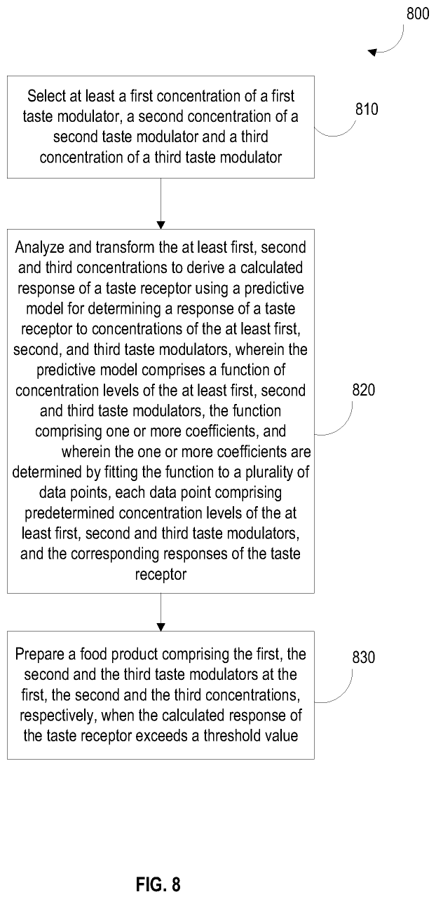

FIG. 8 is a flow diagram that depicts a method 800 for preparing or enhancing palatability of a food product using a predictive model for determining a response of a taste receptor to concentrations of at least a first taste modulator, a second taste modulator and a third taste modulator. The method may begin at step 810, where the computing system can select at least a first concentration of the first taste modulator, a second concentration of the second taste modulator and a third concentration of the third taste modulator. At step 820, the computing system can analyze and transform the at least first, second and third concentrations to derive a calculated response of a taste receptor using the predictive model, wherein the predictive model comprises a function of concentration levels of the at least first, second and third taste modulators, the function comprising one or more coefficients, and wherein the one or more coefficients are determined by fitting the function to a plurality of data points, each data point comprising predetermined concentration levels of the at least first, second and third taste modulators, and the corresponding responses of the taste receptor. At step 830, the computing system can prepare the food product comprising the first, the second and the third taste modulators at the first, the second and the third concentrations, respectively, when the calculated response of the taste receptor exceeds a threshold value. Particular embodiments may repeat one or more steps of the method of FIG. 8, where appropriate. Although this disclosure describes and illustrates particular steps of the method of FIG. 8 as occurring in a particular order, this disclosure contemplates any suitable steps of the method of FIG. 8 occurring in any suitable order. Moreover, although this disclosure describes and illustrates an example method for preparing or enhancing palatability of a food product using a predictive model for determining a response of a taste receptor to concentrations of at least a first taste modulator, a second taste modulator and a third taste modulator including the particular steps of the method of FIG. 8, this disclosure contemplates any suitable method for preparing or enhancing palatability of a food product using a predictive model for determining a response of a taste receptor to concentrations of at least a first taste modulator, a second taste modulator and a third taste modulator including any suitable steps, which may include all, some, or none of the steps of the method of FIG. 8, where appropriate. Furthermore, although this disclosure describes and illustrates particular components, devices, or systems carrying out particular steps of the method of FIG. 8, this disclosure contemplates any suitable combination of any suitable components, devices, or systems carrying out any suitable steps of the method of FIG. 8.

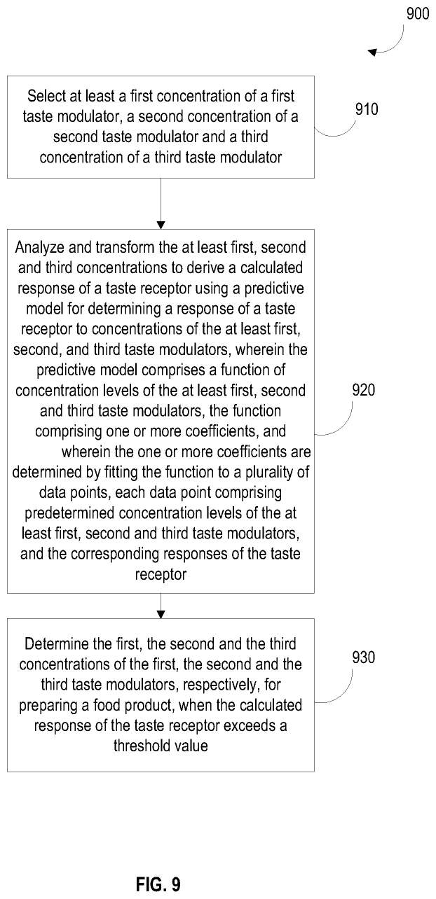

FIG. 9 is a flow diagram that depicts a method 900 for determining concentrations of at least a first taste modulator, a second taste modulator and a third taste modulator in a food product. The method may begin at step 910, where the computing system can select at least a first concentration of the first taste modulator, a second concentration of the second taste modulator and a third concentration of the third taste modulator. At step 920, the computing system can analyze and transform the at least first, second and third concentrations to derive a calculated response of a taste receptor using the predictive model, wherein the predictive model comprises a function of concentration levels of the at least first, second and third taste modulators, the function comprising one or more coefficients, and wherein the one or more coefficients are determined by fitting the function to a plurality of data points, each data point comprising predetermined concentration levels of the at least first, second and third taste modulators, and the corresponding responses of the taste receptor. At step 930, the computing system can determine the first, the second and the third concentrations of the first, the second and the third taste modulators, respectively, for preparing the food product, when the calculated response of the taste receptor exceeds a threshold value. Particular embodiments may repeat one or more steps of the method of FIG. 9, where appropriate. Although this disclosure describes and illustrates particular steps of the method of FIG. 9 as occurring in a particular order, this disclosure contemplates any suitable steps of the method of FIG. 9 occurring in any suitable order. Moreover, although this disclosure describes and illustrates an example method for pet wellness assessment including the particular steps of the method of FIG. 9, this disclosure contemplates any suitable method for pet wellness assessment including any suitable steps, which may include all, some, or none of the steps of the method of FIG. 9, where appropriate. Furthermore, although this disclosure describes and illustrates particular components, devices, or systems carrying out particular steps of the method of FIG. 9, this disclosure contemplates any suitable combination of any suitable components, devices, or systems carrying out any suitable steps of the method of FIG. 9.

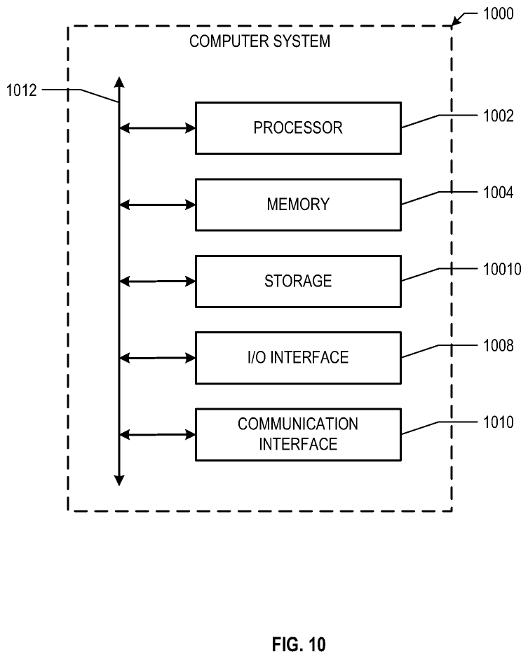

FIG. 10 illustrates an example computer system 1000. In particular embodiments, one or more computer systems 1000 perform one or more steps of one or more methods described or illustrated herein. In particular embodiments, one or more computer systems 1000 provide functionality described or illustrated herein. In particular embodiments, software running on one or more computer systems 1000 performs one or more steps of one or more methods described or illustrated herein or provides functionality described or illustrated herein. Particular embodiments include one or more portions of one or more computer systems 1000. Herein, reference to a computer system may encompass a computing device, and vice versa, where appropriate. Moreover, reference to a computer system may encompass one or more computer systems, where appropriate.

This disclosure contemplates any suitable number of computer systems 1000. This disclosure contemplates computer system 1000 taking any suitable physical form. As example and not by way of limitation, computer system 1000 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. As another example and not by way of limitation, computer system 1000 may be based on other computer system configurations including hand-held devices, microprocessor systems, microprocessor-based or programmable consumer electronics, minicomputers, mainframe computers, and the like. Where appropriate, computer system 1000 may include one or more computer systems 1000;

be unitary or distributed; span multiple locations; span multiple machines; span multiple data centers; or reside in a cloud, which may include one or more cloud components in one or more networks. Where appropriate, one or more computer systems 1000 may perform without substantial spatial or temporal limitation one or more steps of one or more methods described or illustrated herein. As an example and not by way of limitation, one or more computer systems 1000 may perform in real time or in batch mode one or more steps of one or more methods described or illustrated herein. One or more computer systems 1000 may perform at different times or at different locations one or more steps of one or more methods described or illustrated herein, where appropriate.

In particular embodiments, computer system 1000 includes a processor 1002, memory 1004, storage 1006, an input/output (I/O) interface 1008, a communication interface 1010, and a bus 1012. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1002 includes hardware for executing instructions, such as those making up a computer program. As an example and not by way of limitation, to execute instructions, processor 1002 may retrieve (or fetch) the instructions from an internal register, an internal cache, memory 1004, or storage 1006; decode and execute them; and then write one or more results to an internal register, an internal cache, memory 1004, or storage 1006. In particular embodiments, processor 1002 may include one or more internal caches for data, instructions, or addresses. This disclosure contemplates processor 1002 including any suitable number of any suitable internal caches, where appropriate. As an example and not by way of limitation, processor 1002 may include one or more instruction caches, one or more data caches, and one or more translation lookaside buffers (TLBs). Instructions in the instruction caches may be copies of instructions in memory 1004 or storage 1006, and the instruction caches may speed up retrieval of those instructions by processor 1002. Data in the data caches may be copies of data in memory 1004 or storage 1006 for instructions executing at processor 1002 to operate on; the results of previous instructions executed at processor 1002 for access by subsequent instructions executing at processor 1002 or for writing to memory 1004 or storage 1006; or other suitable data. The data caches may speed up read or write operations by processor 1002. The TLBs may speed up virtual-address translation for processor 1002. In particular embodiments, processor 1002 may include one or more internal registers for data, instructions, or addresses. This disclosure contemplates processor 1002 including any suitable number of any suitable internal registers, where appropriate. Where appropriate, processor 1002 may include one or more arithmetic logic units (ALUs); be a multi-core processor; or include one or more processors 1002. Although this disclosure describes and illustrates a particular processor, this disclosure contemplates any suitable processor.

In particular embodiments, memory 1004 includes main memory for storing instructions for processor 1002 to execute or data for processor 1002 to operate on. As an example and not by way of limitation, computer system 1000 may load instructions from storage 1006 or another source (such as, for example, another computer system 1000) to memory 1004. Processor 1002 may then load the instructions from memory 1004 to an internal register or internal cache. To execute the instructions, processor 1002 may retrieve the instructions from the internal register or internal cache and decode them. During or after execution of the instructions, processor 1002 may write one or more results (which may be intermediate or final results) to the internal register or internal cache. Processor 1002 may then write one or more of those results to memory 1004. In particular embodiments, processor 1002 executes only instructions in one or more internal registers or internal caches or in memory 1004 (as opposed to storage 1006 or elsewhere) and operates only on data in one or more internal registers or internal caches or in memory 1004 (as opposed to storage 1006 or elsewhere). One or more memory buses (which may each include an address bus and a data bus) may couple processor 1002 to memory 1004. Bus 1010 may include one or more memory buses, as described below. In particular embodiments, one or more memory management units (MMUs) reside between processor 1002 and memory 1004 and facilitate accesses to memory 1004 requested by processor 1002. In particular embodiments, memory 1004 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1004 may include one or more memories 1004, where appropriate. Although this disclosure describes and illustrates particular memory, this disclosure contemplates any suitable memory.

In particular embodiments, storage 1006 includes mass storage for data or instructions. As an example and not by way of limitation, storage 1006 may include a hard disk drive (HDD), a floppy disk drive, flash memory, an optical disc, a magneto-optical disc, magnetic tape, or a Universal Serial Bus (USB) drive or a combination of two or more of these. Storage 1006 may include removable or non-removable (or fixed) media, where appropriate. Storage 1006 may be internal or external to computer system 1000, where appropriate. In particular embodiments, storage 1006 is non-volatile, solid-state memory. In particular embodiments, storage 1006 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1006 taking any suitable physical form. Storage 1006 may include one or more storage control units facilitating communication between processor 1002 and storage 1006, where appropriate. Where appropriate, storage 1006 may include one or more storages 1006. Although this disclosure describes and illustrates particular storage, this disclosure contemplates any suitable storage.

In particular embodiments, I/O interface 1008 includes hardware, software, or both, providing one or more interfaces for communication between computer system 1000 and one or more I/O devices. Computer system 1000 may include one or more of these I/O devices, where appropriate. One or more of these I/O devices may enable communication between a person and computer system 1000. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1008 for them. Where appropriate, I/O interface 1008 may include one or more device or software drivers enabling processor 1002 to drive one or more of these I/O devices. I/O interface 1008 may include one or more I/O interfaces 1008, where appropriate. Although this disclosure describes and illustrates a particular I/O interface, this disclosure contemplates any suitable I/O interface.

In particular embodiments, communication interface 1010 includes hardware, software, or both providing one or more interfaces for communication (such as, for example, packet-based communication) between computer system 1000 and one or more other computer systems 1000 or one or more networks. As an example and not by way of limitation, communication interface 1010 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1010 for it. As an example and not by way of limitation, computer system 1000 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1000 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1000 may include any suitable communication interface 1010 for any of these networks, where appropriate. Communication interface 1010 may include one or more communication interfaces 1010, where appropriate. Although this disclosure describes and illustrates a particular communication interface, this disclosure contemplates any suitable communication interface.

In particular embodiments, bus 1012 includes hardware, software, or both coupling components of computer system 1000 to each other. As an example and not by way of limitation, bus 1012 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1012 may include one or more buses 1012, where appropriate. Although this disclosure describes and illustrates a particular bus, this disclosure contemplates any suitable bus or interconnect.

One or more embodiments of the present disclosure may be implemented as one or more computer programs or as one or more computer program modules embodied in one or more computer readable media. The term computer readable medium refers to any data storage device that can store data which can thereafter be input to a computer system-computer readable media may be based on any existing or subsequently developed technology for embodying computer programs in a manner that enables them to be read by a computer. Herein, a computer-readable non-transitory storage medium or media may include one or more semiconductor-based or other integrated circuits (ICs) (such as, for example, field-programmable gate arrays (FPGAs) or application-specific ICs (ASICs)), network attached storage (NAS), read-only memory, random-access memory, hard disk drives (HDDs), hybrid hard drives (HHDs), optical discs, optical disc drives (ODDs), CDs (Compact Discs) (such as, for example, CD-ROM, a CD-R, or a CD-RW, a DVD), magneto-optical discs, magneto-optical drives, floppy diskettes, floppy disk drives (FDDs), magnetic tapes, solid-state drives (SSDs), RAM-drives, SECURE DIGITAL cards or drives, other optical and non-optical data storage devices, any other suitable computer-readable non-transitory storage media, or any suitable combination of two or more of these, where appropriate. A computer-readable non-transitory storage medium may be volatile, non-volatile, or a combination of volatile and non-volatile, where appropriate. The computer readable medium can also be distributed over a network coupled computer system so that the computer readable code is stored and executed in a distributed fashion.

Although one or more embodiments of the present disclosure have been described in some detail for clarity of understanding, it will be apparent that certain changes and modifications may be made within the scope of the claims. Accordingly, the described embodiments are to be considered as illustrative and not restrictive, and the scope of the claims is not to be limited to details given herein, but may be modified within the scope and equivalents of the claims. In the claims, elements and/or steps do not imply any particular order of operation, unless explicitly stated in the claims.

Many variations, modifications, additions, and improvements can be made. Plural instances may be provided for components, operations or structures described herein as a single instance. Boundaries between various components, operations and data stores are somewhat arbitrary, and particular operations are illustrated in the context of specific illustrative configurations. Other allocations of functionality are envisioned and may fall within the scope of the disclosure(s). In general, structures and functionality presented as separate components in exemplary configurations may be implemented as a combined structure or component. Similarly, structures and functionality presented as a single component may be implemented as separate components. These and other variations, modifications, additions, and improvements may fall within the scope of the appended claim(s).

### 3. In Vitro Assays

The presently disclosed subject matter further provides in vitro methods for determining the response of a taste receptor to one or more taste modulators at selected concentration levels.

The taste receptors for use in the presently disclosed methods can include isolated or recombinant taste receptors or cells expressing a taste receptor, disclosed herein. In certain embodiments, the taste receptor for use in the disclosed methods can be a human taste receptor, a feline taste receptor and/or a canine taste receptor. In certain embodiments, the taste receptor for use in the disclosed methods can have multiple biding sites. As an example and not by way of limitation, the taste receptor can be an umami taste receptor, a salty taste receptor, a kokumi taste receptor, a bitter taste receptor, a fatty acid taste receptor, a sweet taste receptor and/or a sour taste receptor.

In certain embodiments, the method for determining the response of a taste receptor comprises measuring the biological activity of a taste receptor in the absence and/or presence of one or more taste modulators at selected concentration levels.

In certain embodiments, the one or more taste modulators at selected concentration levels increase the biological activity of a taste receptor by at least about 5%, 10%, 15%, 20%, 25%, 30%, 35%, 40%, 45%, 50%, 55%, 60%, 65%, 70%, 75%, 80%, 85%, 90%, 95%, 100%, or more, compared to the biological activity of the taste receptor when the compound is not present. In certain embodiments, the one or more taste modulators at selected concentration levels increase the biological activity of a taste receptor by at least about 30% compared to the biological activity of the taste receptor when the compound is not present.

In certain embodiments, the method comprises expressing a taste receptor in a cell line and measuring the biological activity of the receptor in the presence and/or absence of one or more taste modulators at selected concentration levels.

In particular embodiments, the computing system can detect activation of the receptor in the disclosed methods using a labelling compound and/or agent. In certain embodiments, the computing system can determine the activity of the taste receptor by the detection of secondary messengers such as, but not limited to, cAMP, cGMP, IP3, DAG or calcium. In certain embodiments, the computing system can determine the activity of the taste receptor by the detection of the intracellular calcium levels. Monitoring can be by way of luminescence or fluorescence detection, such as by a calcium sensitive fluorescent dye. In certain embodiments, the computing system can determine intracellular calcium levels using a cellular dye, e.g., a fluorescent calcium indicator such as Calcium 4. In certain embodiments, the computing system can determine intracellular calcium levels by measuring the level of calcium binding to a calcium-binding protein, for example, calmodulin. Alternatively and/or additionally, the computing system can determine activity of the taste receptor by detection of the phosphorylation, transcript levels and/or protein levels of one or more downstream protein targets of the taste receptor.

The cell line used in the disclosed methods can include any cell type that is capable of expressing a taste receptor. Non-limiting examples of cells that can be used in the disclosed methods include HeLa cells, Chinese hamster ovary cells (CHO cells), African green monkey kidney cells (COS cells), Xenopus oocytes, HEK-293 cells and murine 3T3 fibroblasts. In certain embodiments, the method can include expressing a taste receptor in CHO-K1 cells. In certain embodiments, the method can include expressing a taste receptor in HEK-293 cells. In certain embodiments, the method can include expressing a taste receptor in COS cells. In certain embodiments, the cells constitutively express the taste receptor. In another embodiment, expression of the taste receptor by the cells is inducible.

In certain embodiments, the cell expresses a calcium-binding photoprotein, wherein the photoprotein luminesces upon binding calcium. In certain embodiments, the calcium binding photoprotein comprises the protein clytin. In certain embodiments the clytin is a recombinant clytin. In certain embodiments, the clytin comprises an isolated clytin, for example, a clytin isolated from Clytia gregarium. In certain embodiments, the calcium-binding photoprotein comprises the protein aequorin, for example, a recombinant aequorin or an isolated aequorin, such as an aequorin isolated from Aequorea victoria. In certain embodiments, the calcium-binding photoprotein comprises the protein obelin, for example, a recombinant obelin or an isolated obelin, such as an obelin isolated from Obelia longissima.

In certain embodiments, expression of a taste receptor in a cell can be performed by introducing a nucleic acid encoding a taste receptor into the cell. In certain embodiments, the introduction of a nucleic acid into a cell can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92 (1985), the disclosures of which are hereby incorporated by reference in their entireties) and can be used in accordance with the disclosed subject matter. In certain embodiments, the technique can provide for stable transfer of nucleic acid to the cell, so that the nucleic acid is expressible by the cell and inheritable and expressible by its progeny. In certain embodiments, the technique can provide for a transient transfer of the nucleic acid to the cell, so that the nucleic acid is expressible by the cell, wherein heritability and expressibility decrease in subsequent generations of the cell's progeny.

In certain non-limiting embodiments, the in vitro assay comprises cells expressing a taste receptor that is native to the cells. Examples of such cells expressing a native taste receptor include, for example but not limited to, human, dog (canine) and/or cat (feline) taste cells (e.g., primary taste receptor cells). In certain embodiments, the human, dog and/or cat taste cells expressing a taste receptor are isolated from a human, dog and/or cat and cultured in vitro. In certain embodiments, the taste receptor cells can be immortalized, for example, such that the cells isolated from a dog and/or cat can be propagated in culture.

In certain embodiments, expression of a taste receptor in a cell can be induced through gene editing, for example, through use of the CRISPR gene editing system to incorporate a taste receptor gene into the genome of a cell, or to edit or modify a taste receptor gene native to the cell.

In certain embodiments, the method can further include analyzing two or more, three or more or four or more test compounds in combination. In certain embodiments, the two or more, three or more or four or more test compounds can be from different classes of compounds, e.g., amino acids, small chemical compounds, and/or protein hydrolysates.

In certain embodiments, the method for identifying compounds that modulate the activity and/or expression of a receptor comprises determining whether a compound modulates the receptor directly, for example, as an agonist or antagonist. In certain embodiments, the method comprises determining whether a compound indirectly modulates the activity of the receptor (e.g., as an allosteric modulator), for example, by enhancing or decreasing the effect of other compounds on activating or inhibiting receptor activity.

In certain embodiments, the method for identifying compounds that modulate the activity and/or expression of a receptor comprises expressing a receptor in a cell line and measuring the biological activity of the receptor in the presence and/or absence of a test compound. The method can further comprise identifying test compounds that modulate the activity of the receptor by determining if there is a difference in receptor activation in the presence of a test compound compared to the activity of the receptor in the absence of the test compound. In certain embodiments, the selectivity of the putative modulator can be evaluated by comparing its effects on taste receptors, e.g., umami, fatty acid, TIR, CaSR, etc. receptors. Activation of the receptor in the disclosed methods can be detected through the use of a labeling compound and/or agent. In certain embodiments, the activity of a receptor can be determined by the detection of secondary messengers such as, but not limited to, cAMP, cGMP, IP3, DAG or calcium. In certain embodiments, the activity of the receptor can be determined by the detection of the intracellular calcium levels. Monitoring can be by way of luminescence or fluorescence detection, such as by a calcium sensitive fluorescent dye. In certain embodiments, the intracellular calcium levels can be determined using a cellular dye, e.g., a fluorescent calcium indicator such as Calcium 4. In certain non-limiting embodiments, the calcium sensitive fluorescent dye is selected from the group consisting of Fura-2 AM, Fura-2 pentapotassium, Fura Red AM, Indo-1 AM, Indo-1 pentapotassium, Fluo-3, Fluo-4, Fluo-8, Calcium Green-1, Calcium 3, Calcium 4, Calcium 5, Rhod-2, derivatives thereof and combinations thereof. In certain embodiments, the intracellular calcium levels can be determined by measuring the level of calcium binding to a calcium-binding protein, for example, calmodulin. Alternatively, and/or additionally, activity of the receptor can be determined by detection of the phosphorylation, transcript levels and/or protein levels of one or more downstream protein targets of the receptor.

In certain embodiments, the interaction between a test agent and one or more amino acids in the receptor is determined by site directed mutagenesis, x-ray crystallography, x-ray spectroscopy, Nuclear Magnetic Resonance (NMR), cross-linking assessment, mass spectroscopy, electrophoresis, displacement assay, and combinations thereof.

In certain embodiments, the receptor is expressed by a cell, and wherein the test agent is contacted to the cell.

In certain embodiments, the cell expresses a calcium-binding photoprotein. In certain embodiments, the calcium-binding photoprotein is selected from the group consisting of clytin, aequorin, obelin, any recombinant or isolated versions thereof, and any combinations thereof.

In certain embodiments of the methods disclosed, the activity of the receptor is determined by monitoring an intracellular calcium level by a luminescence detection or a fluorescence detection. In specific embodiments, the fluorescence detection comprises a calcium sensitive fluorescent dye selected from the group consisting of Fura-2 AM, Fura-2 pentapotassium, Fura Red AM, Indo-1 AM, Indo-1 pentapotassium, Fluo-3, Fluo-4, Fluo-8, Calcium Green-1, Calcium 3, Calcium 4, Calcium 5, Rhod-2, derivatives thereof and combinations thereof.

In certain embodiments of the disclosed method, the receptor is expressed by a cell, and the test agent is contacted to the cell. In specific embodiments, the cell expresses a calcium-binding photoprotein. In certain embodiments, the calcium-binding photoprotein is selected from the group consisting of clytin, aequorin, obelin, any recombinant or isolated versions thereof, and any combinations thereof.

In certain embodiments of the disclosed method, the method for determining the activity of the receptor comprises monitoring an intracellular calcium level by a luminescence detection or a fluorescence detection.

### EXAMPLES

The presently disclosed subject matter will be better understood by reference to the following Examples, which are provided as exemplary of the invention, and not by way of limitation.

### Example 1: Modelling of GMP, Alanine and Benzyl-L-Phenylalanine Methyl Ester HCl Activation of the Cat Umami Receptor with Different Models and Selecting a Model with Top Performance from these Models

The present example describes the modelling of cat umami receptor activation by a combination of the compounds GMP, alanine and Benzyl-L-phenylalanine methyl ester HCl.

Methods: In accordance with the techniques described above, the generation of three models for combining different concentrations of GMP, alanine, and Benzyl-L-phenylalanine methyl ester HCl is described. First, the computing system generated a predictive model based on the Hill equation. The model was generated based on experimental data comprising 768 data points, representing 8 concentrations of Benzyl-L-phenylalanine methyl ester HCl, 12 concentrations of GMP, and 8 concentrations of alanine. The concentration of Benzyl-L-phenylalanine methyl ester HCl was varied from 0 to 1 mM, the concentration of GMP was varied from 0 to 1 mM, and the concentration of alanine was varied from 0 to 100 mM. The Hill equation that most closely approximated the experimental data had coefficients aₓ=0, a_{y}=0, a_{z}=2.68, a_{xy}=0.09, a_{yz}=0.07, a_{xz}=12, a_{xyz}=5, and n=5.

Next, a predictive model based on the logistic function was generated based on the same experimental data. The logistic function that most closely approximated the experimental data had coefficients aₓ=0, a_{y}=0, a_{z}=0, a_{xy}=0, a_{yz}=0, a_{xz}=5, a_{xyz}=10, slope a=2.2, and offset b=0.7.

Lastly, a predictive model based on the hyperbolic tangent function was generated based on the same experimental data. The hyperbolic tangent function that most closely approximated the experimental data had coefficients aₓ=0, a_{y}=0, a_{z}=0, a_{xy}=0, a_{yz}=0.1, a_{xz}=10, a_{xyz}=5, slope a=2.5, and scaling factor b=0.8.

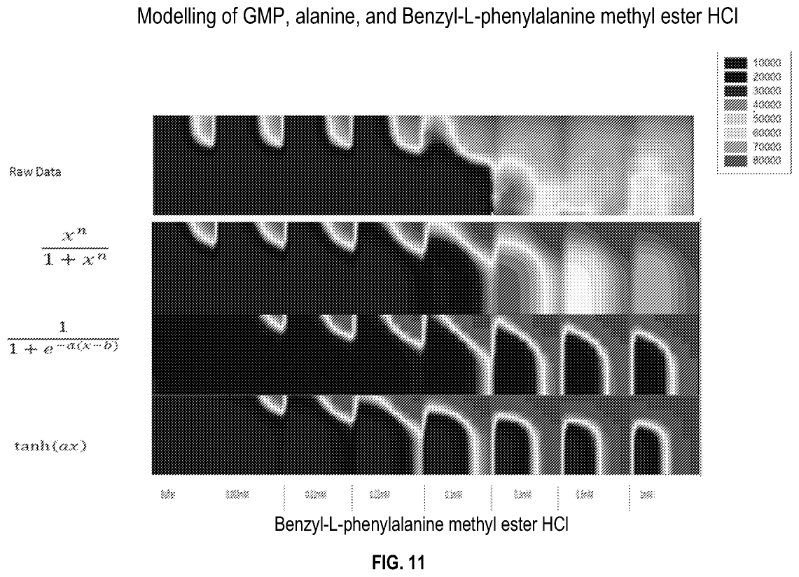

Results: FIG. 11 is a visual comparison of the modeling results of the three sigmoid functions with experimental results of applying varying concentrations of three taste modulators to the umami receptor of a cat, where the same concentrations of taste modulators are input to the model. The results depicted are for 8 concentrations of benzyl-1-phenylalanine methyl ester HCl, where, for each concentration of benzyl-1-phenylalanine methyl ester HCl, the concentration of GMP is varied from 0 to 1 mM, while the concentration of alanine is varied from 0 to 100 mM. The sigmoid functions are the Hill equation, the logistic function, and the hyperbolic tangent function. As shown in the figure, for the given concentrations, the Hill equation most closely approximates the experimental data, with an error rate of approximately 10 percent, while the logistic and hyperbolic tangent functions have an error rate of approximately 25 percent. Therefore, in accordance with method 300, the computing system would select the Hill equation as the preferred model for predicting the receptor response for combinations of the aforementioned compounds.

### Example 2: Modelling of GMP, Alanine and n-(heptan-4-yl)benzo[d][1,3]dioxole-5-carboxamide Activation of the Cat Umami Receptor

The present example describes the modelling of cat umami receptor activation by a combination of the compounds GMP, alanine and N-(heptan-4-yl)benzo[d][1,3]dioxole-5-carboxamide.

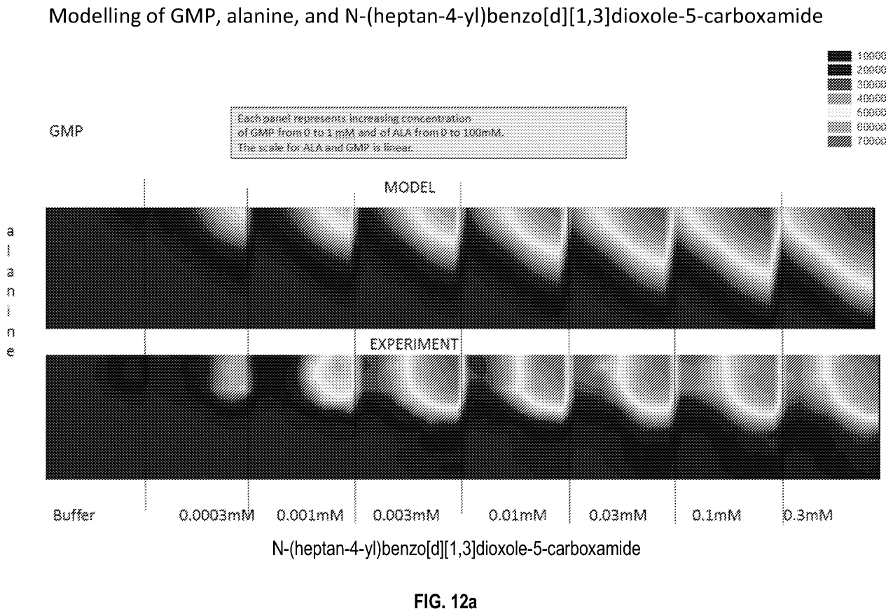

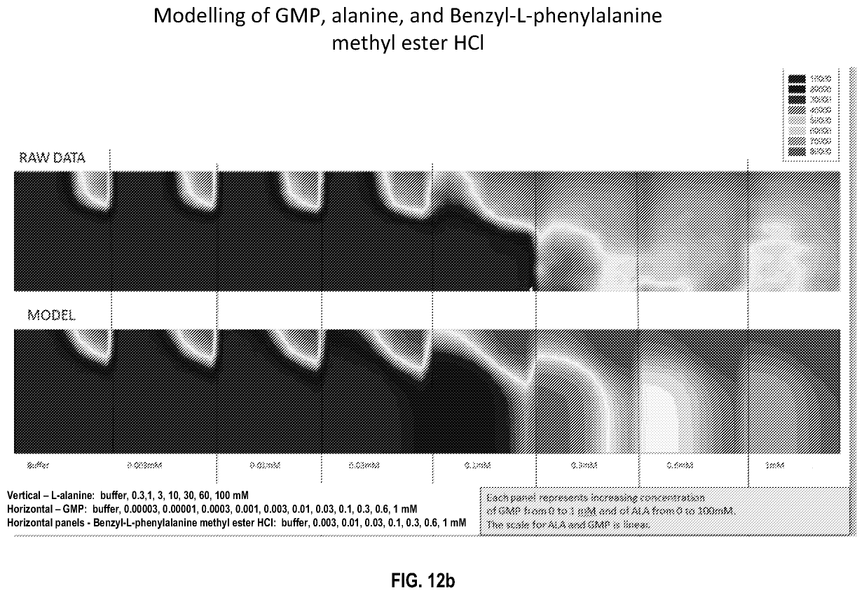

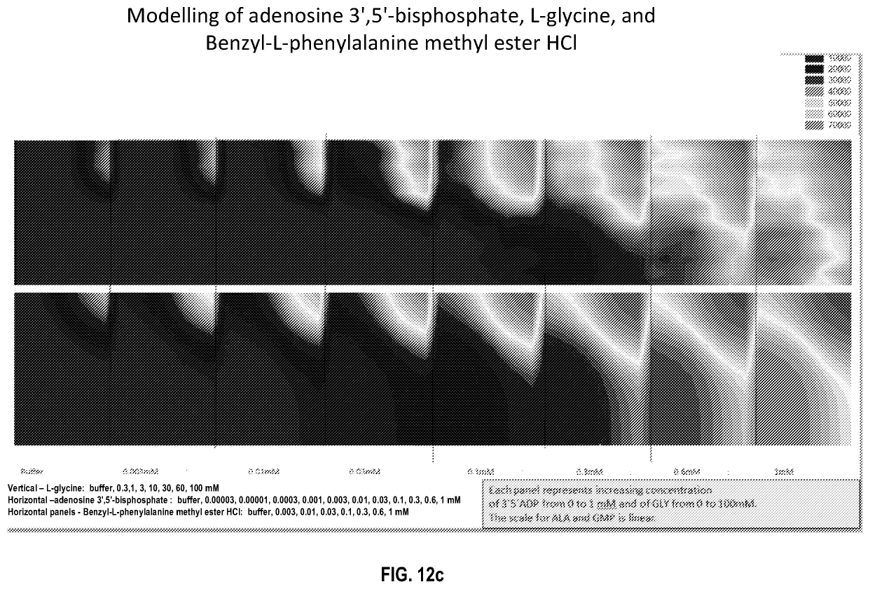

Methods: In accordance with the techniques described above, the computing system generated a Hill equation-based model to model combinations of the compounds GMP, alanine and N-(heptan-4-yl)benzo[d][1,3]dioxole-5-carboxamide. The experimental data comprises 768 data points (i.e., 8 concentrations of N-(heptan-4-yl)benzo[d][1,3]dioxole-5-carboxamide, 12 concentrations of GMP, and 8 concentrations of alanine), where GMP was varied in concentration from 0 to 1 mM, alanine was varied from 0 to 100 mM, and N-(heptan-4-yl)benzo[d][1,3]dioxole-5-carboxamide was varied from 0 to 0.3 mM. In this example, the Hill equation most closely approximates the experimental data, as described below with reference to FIG. 12a.

Results: FIG. 12a shows a comparison of experimental and model results for 8 concentrations of N-(heptan-4-yl)benzo[d][1,3]dioxole-5-carboxamide, where, for each concentration of N-(heptan-4-yl)benzo[d][1,3]dioxole-5-carboxamide, the concentration of GMP was varied from 0 to 1 mM, while the concentration of alanine was varied from 0 to 100 mM. In this case, the Hill equation approximates the experimental data with the lowest rate of error. The coefficients are as follows: aₓ=0, a_{y}=0, a_{z}=0, a_{xy}=0, a_{yz}=0, a_{xz}=2.1, a_{xyz}=78.1, and n=0.311, which gave rise to a sigmoid function represented by the equation

$$f ( x , y , z ) = \frac{{( 2.1 xz + 78.1 xyz )}^{0.311}}{1 + {( 2.1 xz + 78.1 xyz )}^{0.311}} .$$

### Example 3: Modelling of GMP, Alanine and Benzyl-L-Phenylalanine Methyl Ester HCl Activation of the Cat Umami Receptor with a Hill Equation-Based Model

The present example describes the modelling of cat umami receptor activation by a combination of the compounds GMP, alanine and Benzyl-L-phenylalanine methyl ester HCl.

Methods: In accordance with the techniques described above, the computing system generated a Hill equation-based model to model combinations of the compounds GMP, alanine and Benzyl-L-phenylalanine methyl ester HCl. The experimental data comprises 768 data points (i.e., 8 concentrations Benzyl-L-phenylalanine methyl ester HCl, 12 concentrations of GMP, and 8 concentrations of alanine), where GMP was varied in concentration from 0 to 1 mM, alanine was varied from 0 to 100 mM, and Benzyl-L-phenylalanine methyl ester HCl was varied from 0 to 1 mM. In this example, the Hill equation most closely approximated the experimental data, as described below with reference to FIG. 12b.

Results: FIG. 12b shows a comparison of experimental and model results for 8 concentrations of benzyl-1-phenylalanine methyl ester HCl, where, for each concentration of benzyl-1-phenylalanine methyl ester HCl, the concentration of GMP was varied from 0 to 1 mM, while the concentration of alanine was varied from 0 to 100 mM. In this case, the Hill equation approximated the experimental data with the lowest rate of error. The coefficients are as follows: aₓ=0, a_{y}=0, a_{z}=2.68, a_{xy}=0.09, a_{yz}=0.07 a_{xz}=12, a_{xyz}=5, and n=1.41, which gives rise to a sigmoid function represented by the equation

$$f ( x , y , z ) = \frac{{( 2.68 z + 0.09 xy + 0.07 yz + 12 xz + 5 xyz )}^{1.41}}{1 + {( 2.68 z + 0.09 xy + 0.07 yz + 12 xz + 5 xyz )}^{1.41}} .$$

### Example 4: Modelling of Adenosine 3′,5′-Bisphosphate (ADP), Glycine and Benzyl-L-Phenylalanine Methyl Ester HCl Activation of the Cat Umami Receptor

The present example describes the modelling of cat umami receptor activation by a combination of the compounds 3′,5′-bisphosphate (ADP), glycine and Benzyl-L-phenylalanine methyl ester HCl.

Methods: In accordance with the techniques described above, the computing system generated a Hill equation-based model to model combinations of the compounds adenosine 3′,5′-bisphosphate (ADP), glycine and Benzyl-L-phenylalanine methyl ester HCl. The experimental data comprises 768 data points (i.e., 8 concentrations Benzyl-L-phenylalanine methyl ester HCl, 12 concentrations of adenosine 3′,5′-bisphosphate (ADP), and 8 concentrations of glycine), where 3′,5′-bisphosphate (ADP) was varied in concentration from 0 to 1 mM, glycine was varied from 0 to 100 mM, and Benzyl-L-phenylalanine methyl ester HCl was varied from 0 to 1 mM. In this example, the Hill equation most closely approximated the experimental data, as described below with reference to FIG. 12c.

Results: FIG. 12c shows a comparison of experimental and model results for 8 concentrations of benzyl-1-phenylalanine methyl ester HCl, where, for each concentration of benzyl-1-phenylalanine methyl ester HCl, the concentration of adenosine 3′,5′-bisphosphate was varied from 0 to 1 mM, while the concentration of L-glycine was varied from 0 to 100 mM. In this case, the Hill equation approximated the experimental data with the lowest rate of error. The coefficients are as follows: aₓ=0, a_{y}=0, a_{z}=0.69, a_{xy}=0.02, a_{yz}=0.07 a_{xz}=5, a_{xyz}=8, and n=0.595, which gave rise to a sigmoid function represented by the equation

$$f ( x , y , z ) = \frac{{( 0.69 z + 0.02 xy + 0.07 yz + 5 xz + 8 xyz )}^{0.595}}{1 + {( 2.68 z + 0.09 xy + 0.07 yz + 12 xz + 5 xyz )}^{0.595}} .$$

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A method of generating a model for determining a response of a taste receptor to concentrations of at least a first taste modulator, a second taste modulator and a third taste modulator, the method comprising, by one or more computing systems:
accessing a plurality of data points, each data point comprising at least one concentration level of the first taste modulator, one concentration level of the second taste modulator and one concentration level of the third taste modulator;
for each data point, determining a corresponding response of the taste receptor to the at least first, second and third taste modulators at the concentration levels according to each data point;
selecting, for the model, a function of the concentration levels of the at least first, second and third taste modulators, the function comprising one or more unknown coefficients; and
determining values for the one or more unknown coefficients by fitting the function to the plurality of data points and the corresponding responses of the taste receptor; and
generating the model based on the function based on the determined values for the one or more unknown coefficients.

2. A method of preparing or enhancing palatability of a food product using a predictive model for determining a response of a taste receptor to concentrations of at least a first taste modulator, a second taste modulator and a third taste modulator, the method comprising, by one or more computing systems:
selecting at least a first concentration of the first taste modulator, a second concentration of the second taste modulator and a third concentration of the third taste modulator;
analyzing and transforming the at least first, second and third concentrations to derive a calculated response of a taste receptor using the predictive model,
wherein the predictive model comprises a function of concentration levels of the at least first, second and third taste modulators, the function comprising one or more coefficients, and
wherein the one or more coefficients are determined by fitting the function to a plurality of data points, each data point comprising predetermined concentration levels of the at least first, second and third taste modulators, and the corresponding responses of the taste receptor; and
preparing the food product comprising the first, the second and the third taste modulators at the first, the second and the third concentrations, respectively, when the calculated response of the taste receptor exceeds a threshold value.

3. The method of claim 1, wherein the corresponding response of the taste receptor is determined by an in vitro assay.

4. The method of claim 3, wherein the in vitro assay comprises contacting the taste receptor with the at least first, second and third taste modulators at the concentration levels according to each data point and detecting a biological activity of the taste receptor.

5. The method of claim 2, wherein the taste receptor is a human taste receptor, a feline taste receptor and/or a canine taste receptor.

6. The method of claim 2, wherein the taste receptor is an umami taste receptor, a kokumi taste receptor, and/or a sweet taste receptor.

7. The method of claim 2, wherein the function is a sigmoid function.

8. The method of claim 2, wherein the function is a logistic function, a Gompetz sigmoid function, a trigonometric function, and/or a Hill equation.

9. The method of claim 2, wherein the one or more coefficients are determined by using nonlinear regression.

10. The method of claim 2, wherein the function has an approximation error of no more than 20%.

11. A food product prepared according to method as recited in claim 2.

12. The food product of claim 11, wherein the food product is a human food product.

13. The food product of claim 12, wherein the pet food product is a feline pet food product or a canine pet food product.

14. The food product of claim 13, wherein the pet food product is a wet pet food product.

15. The food product of claim 14, wherein the pet food product is a dry pet food product.

16.-35. (canceled)

36. The food product of claim 11, wherein the food product is a pet food product.

37. The food product of claim 11, wherein the corresponding response of the taste receptor is determined by an in vitro assay.

38. The food product of claim 37, wherein the in vitro assay comprises contacting the taste receptor with the at least first, second and third taste modulators at the concentration levels according to each data point and detecting a biological activity of the taste receptor.

39. The food product of claim 11, wherein the taste receptor is a human taste receptor, a feline taste receptor and/or a canine taste receptor.

40. The food product of claim 11, wherein the taste receptor is an umami taste receptor, a kokumi taste receptor, and/or a sweet taste receptor.
